(19) United States
(12) Patent Application Publication
(10) Pub. No.: US 2019/0209608 A1
(43) Pub. Date: Jul. 11, 2019

(54) DRINKING WATER COMPOSITION FOR IMPROVING METABOLISM

(71) Applicant: AQUA LOHAS WATER-TECH SERVICE CO., LTD., Miaoli County (TW)

(72) Inventors: JEN-CHUNG CHEN, Miaoli County (TW); CHIH-HUNG LIN, Miaoli County (TW)

(21) Appl. No.: 15/863,467

(22) Filed: Jan. 5, 2018

(51) Int. Cl. A61K 33/30; A61K 33/06; A61K 33/00; A61K 33/24; A61K 33/22; A61K 33/04; A61K 33/18

(52) U.S. Cl. CPC A61K 33/30; A61K 33/06; A61K 33/00; A61K 33/18; A61K 33/22; A61K 33/04; A61K 33/24

(57) ABSTRACT

A drinking water composition for improving the metabolism comprises magnesium, sodium, potassium, calcium and at least one of trace elements selected from the group consisting of chromium, boron, silicon, zinc, selenium, molybdenum, iodine and combinations thereof, in specific proportions. It is experimentally found that the drinking water composition can not only activate the expression of blood glucose regulation genes, but also has a positive impact on the fat storage capacity of cells, and can achieve the effect of improving the metabolism.

### FIELD OF THE INVENTION

The present invention relates to a drinking water composition, and more particularly to a drinking water composition for improving a metabolic effect.

### BACKGROUND OF THE INVENTION

Water is an important part of all living organisms. For example, water accounts for 70% in the human body, which not only participates in biochemical reactions, but also participates in nutrient and oxygen transportation in the body, and further contributes greatly to maintaining the temperature of living organisms.

Aware of the importance of water, the quality of water in daily intake is also taken seriously, and then, researches related to drinking water technologies have become increasingly popular. For example, US Patent Publication No. 2014/0166577 discloses an apparatus and method for producing potable water. In addition to using a reverse osmosis membrane to remove impurities in tap water, a flow rate control unit is used to control the flow rate of wastewater during the process of producing water and cleaning the reverse osmosis membrane, such that the yield of osmosis water is increased stably, and the life of the reverse osmosis membrane is caused to be longer. Except for filtering out impurities, in the present application, a silver ion adding unit and a mineral adding unit for adding minerals to osmosis water are used for adding silver ions and minerals to drinking water, giving the drinking water an antimicrobial property and allowing drinking water to become the so-called “good drink water”.

However, the prior art focuses on the filtration of impurities, or the addition of minerals such as those described above to increase the mineral content of water. However, few studies have explored how the drinking water composition may have a positive impact on the human body.

### SUMMARY OF THE INVENTION

A main objective of the present invention is to solve the disadvantage that the drinking water produced by the conventional drinking water technology can not specifically provide a positive impact on the human body.

In order to achieve said objective, the present invention provides a drinking water composition for improving the metabolism, which is mainly composed of water and comprises the following components: magnesium in a weight percentage ranging from 75,000 ppm to 91,000 ppm; sodium in a weight percentage ranging from 1,000 ppm to 8,000 ppm; potassium in a weight percentage ranging from 500 ppm to 8,000 ppm; calcium in a weight percentage ranging from 20 ppm to 65,000 ppm; and at least one of trace elements selected from a group consisting of chromium, boron, silicon, zinc, selenium, molybdenum, iodine, and combinations thereof, wherein each of the trace elements independently has a weight percentage ranging from 100 ppb to 100 ppm.

The present invention provides a use of a drinking water composition in improving the metabolism. The drinking water composition is mainly composed of water and comprises the following components: magnesium in a weight percentage ranging from 75,000 ppm to 91,000 ppm; sodium in a weight percentage ranging from 1,000 ppm to 8,000 ppm; potassium in a weight percentage ranging to 500 ppm to 8,000 ppm; calcium in a weight percentage ranging from 20 ppm to 65,000 ppm; and at least one of trace elements selected from a group consisting of chromium, boron, silicon, zinc, selenium, molybdenum, iodine, and combinations thereof, wherein each of the trace elements independently has a weight percentage ranging from 100 ppb to 100 ppm.

Therefore, compared with the prior art, the drinking water composition for improving the metabolism provided by the present invention can not only activate the expression of blood glucose regulation genes, have a positive impact on the fat storage capacity of cells, and achieve the effect of improving the metabolism, because of comprising magnesium, sodium, potassium, calcium and at least one of trace elements selected from the group consisting of chromium, boron, silicon, zinc, selenium, molybdenum, iodine and combinations thereof, in specific proportions.

### BRIEF DESCRIPTION OF THE DRAWINGS

FIG. 1 is the activity test result of 3T3-L1 pre-adipocytes treated with the first embodiment of the present invention.

FIG. 2 is the activity test result of 3T3-L1 pre-adipocytes treated with the second embodiment of the present invention.

FIG. 3 is the differentiation result of 3T3-L1 pre-adipocytes treated with the first embodiment and the second embodiment of the present invention into the adipocytes.

FIG. 4 is the results that PPARα, PPARγ, and GLUT4 are activated after the 3T3-L1 pre-adipocytes and adipocytes respectively treated with the first embodiment and the second embodiment of the present invention.

### DETAILED DESCRIPTION OF THE PREFERRED EMBODIMENTS

The detailed description and the technical content of the present invention will now be described with reference to the accompanying drawings as follow.

The present invention provides a drinking water composition for improving the metabolism, which is mainly composed of water and comprises magnesium, sodium, potassium, calcium and at least one of trace elements selected from the group consisting of chromium, boron, silicon, zinc, selenium, molybdenum, iodine, and combinations thereof.

Specifically, in the drinking water composition provided by the present invention, a weight percentage of magnesium ranges from 75,000 ppm to 91,000 ppm; a weight percentage of sodium ranges from 1,000 ppm to 8,000 ppm; a weight percentage of potassium ranges from 500 ppm to 8,000 ppm; a weight percentage of calcium ranges from 20 ppm to 65,000 ppm; and a weight percentages of each of at least one of trace elements selected from a group consisting of chromium, boron, silicon, zinc, selenium, molybdenum, iodine, and combinations thereof independently ranges from 100 ppb to 100 ppm.

In an embodiment of the present invention, the trace elements is preferably selected from a group consisting of chromium, zinc, selenium and combinations thereof, for example, chromium and zinc, chromium and selenium, zinc and selenium, as well as chromium, zinc and selenium. The weight percentage of each of the trace elements in the drinking water composition for improving the metabolism ranges from 200 ppb to 25 ppm independently. For example, in an embodiment of the present invention, the drinking water composition for improving the metabolism comprises the following components: magnesium in a weight percentage ranging from 75,000 ppm to 91,000 ppm; sodium in a weight percentage ranging from 1,000 ppm to 8,000 ppm; potassium in a weight percentage ranging from 500 ppm to 8,000 ppm; calcium in a weight percentage ranging from 20 ppm to 65,000 ppm; two trace elements, i.e., zinc in a weight percentage ranging from 200 ppb to 1 ppm, and selenium in a weight percentage ranging from 8 ppm to 25 ppm.

The drinking water composition for improving the metabolism according to the present invention preferably has magnesium hardness ranging from 250 mg/L to 5,000 mg/L, and more preferably ranging from 1,000 mg/L to 3,000 mg/L, and the magnesium hardness is defined as magnesium ion concentration×4.1.

The drinking water composition for improving the metabolism according to the present invention is prepared by concentrating deep sea water having a depth below the sea level ranging from 200 m to 1,500 m, and more preferably from 300 m to 700 m. Furthermore, the preparation method of the prepared drinking water composition for improving the metabolism is not particularly limited, as long as the types and weight percentages of the elements contained in the drinking water composition for improving the metabolism accord with the patent application scope defined by the present invention. For example, the drinking water composition may be prepared by a mixing system. The mixing system may comprise a concentrated solution output device, a purified water output device and a solution mixing device. The drinking water composition may be obtained by mixing a concentrated solution of the drinking water for improving the metabolism in the concentrated solution output device and purified water supplied by the purified water output device in the solution mixing device according to actual demands. In order to obtain the higher-quality concentrated solution of the drinking water for improving the metabolism, the mixing system may further comprise a filtering unit which is disposed in the concentrated solution output device, and/or the purified water output device, and/or the solution mixing device, and the filtering unit may comprise a nano-filtration film.

It has been found experimentally that the drinking water composition for improving the metabolism according to the present invention has the potential to lower blood glucose by activating PPARα, PPARγ and GLUT4 simultaneously, may also promote the differentiation of an adipocyte and has the effect of increasing the fat storage capacity of the adipocyte.

The effects of the drinking water composition for improving the metabolism in the first embodiment and the second embodiment of the present invention on the activity of the adipocyte, the fat storage capacity of the adipocyte and the glucose metabolism capability will be further studied as below experimentally. The compositions and proportions of the first embodiment and the second embodiment are shown in Table 1 below:

TABLE 1

| (Unit: ppb) | First Embodiment | Second Embodiment |
|---|---|---|
| Magnesium | 79,966,000 | 88,186,000 |
| Sodium | 2,700,000 | 1,600,000 |
| Potassium | 2,550,000 | 1,140,000 |
| Calcium | 40,500,000 | 61,500,000 |
| Chromium | 320 | 420 |
| Zinc | 690 | 800 |
| Selenium | 17,000 | 21,000 |

### Experimental Example 1: The Drinking Water Composition for Improving the Metabolism According to the Present Invention Promotes the Activities of a Pre-Adipocyte and an Adipocyte

In order to investigate the effect of the drinking water composition for improving the metabolism according to the present invention on the activity of the adipocyte, in Experimental Example 1, a 3T3-L1 pre-adipocyte and a 3T3-L1 adipocyte are used to analyze the effect of the drinking water composition for improving the metabolism on the activities of the above cells.

First, in the group of the 3T3-L1 pre-adipocyte, the 3T3-L1 pre-adipocyte is first inoculated in a 24-well plate containing a culture solution. After the 3T3-L1 pre-adipocyte is sufficiently adhered to the 24-well plate overnight, the drinking water composition for improving the metabolism in the first embodiment and the second embodiment are added to the 24-well plate respectively to treat the 3T3-L1 pre-adipocyte for 24 hours, 48 hours, and 72 hours, and the 3T3-L1 pre-adipocyte is collected and analyzed at the above time points.

Specifically, at the time points of 24 hours, 48 hours, and 72 hours, the above “analysis” is performed by first washing with phosphate buffered saline (PBS) to remove the supernatant; adding 270 μL of Dulbecco's Modified Eagle Medium (DMEM) and 30 μL of MTT (3-(4,5-Dimethylthiazol-2-yl)-2,5-diphenyl Tetrazolium Bromide, 500 g/mL) reagent to each 24-well plate containing the 3T3-L1 pre-adipocyte, and culturing at 37° C. for 2 hours, during which, succinatedehydrogenase (SDH) in mitochondria of the living cell enables MTT to be reduced into fat-soluble, blue-violet formazan crystals and deposited in the cells; removing the supernatant, and then adding isopropanol (400 μL) till the formazan crystals are dissolved; taking 200 μL of solution from the dissolved product and adding to a 96-well plate; measuring the absorbance of the solution at a wavelength of O.D. 595 nm using an ELISA Reader.

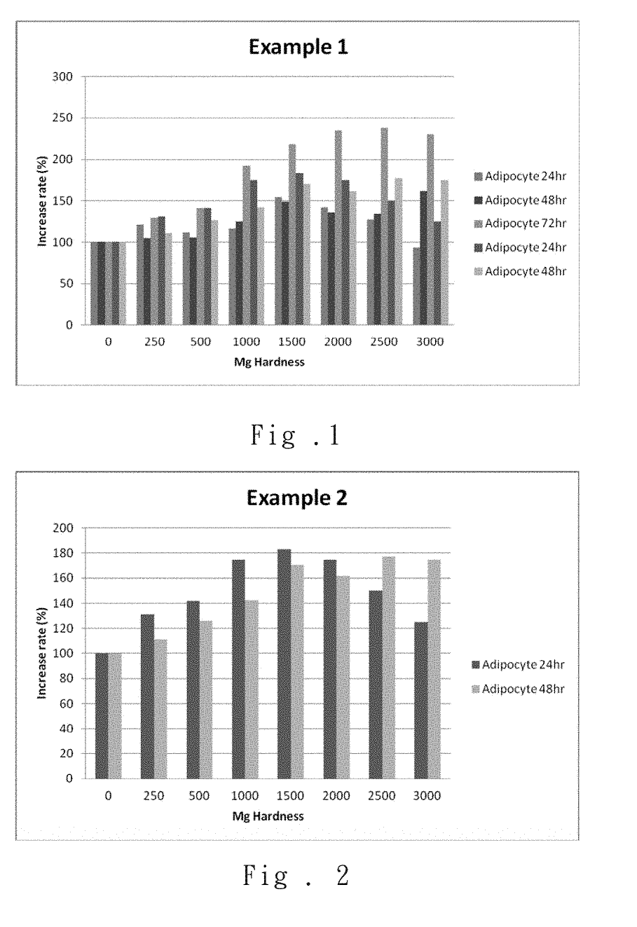

FIG. 1 and FIG. 2 respectively show the activity test results of the 3T3-L1 pre-adipocyte after being treated with the first embodiment and the second embodiment of the present invention, in which the X axis represents magnesium hardness, and the Y axis represents a cell activity increasing rate (%). First, with respect to the 3T3-L1 pre-adipocyte treated for 24 hours with the drinking water composition for improving the metabolism in the first embodiment of the present invention, when the magnesium hardness in the first embodiment exceeds that of a group having a hardness of 1,500 mg/L, it can be observed that the 3T3-L1 adipocyte has a slight increase of activity compared to the experimental group having magnesium hardness of less than 1,500 mg/L, and has an optimal adipocyte activity increase effect when the magnesium hardness ranges from 1, 500 to 3,000 mg/L, as the treatment time increases to 48 hours. This trend is most obvious as the treatment time increases to 72 hours.

It is to be noted that the “magnesium hardness” described in the embodiment of the present invention is defined as the magnesium ion concentration×4.1.

Next, the second embodiment is tested in the same manner as the above. After 24 hours of treatment, it can be observed in the group having a hardness of 250 mg/L that the 3T3-L1 adipocyte has the activity slightly higher than that of an experimental group having a hardness of less than 250 mg/L. However, when the hardness ranges from 1,000 to 3,000 mg/L, said activity of the 3T3-L1 adipocyte will increase more significantly.

The increasing trend of the above-mentioned activity is more obvious as the treatment time increases to 48 hours, and the result is similar to the trend of the first embodiment.

### Experimental Example 2: The Drinking Water Composition for Improving the Metabolism According to the Present Invention Promotes the Differentiation of the Pre-Adipocyte

In Experimental Example 2, the 3T3-L1 pre-adipocyte is treated with the drinking water compositions for improving the metabolism in the first embodiment and the second metabolism first, then added with 250 mM Isobutylmethylxanthine (IBMX), 1 mM DEX and 670 nM insulin and treated for three days, so as to promote the 3T3-L1 pre-adipocyte to be differentiated into an adipocyte.

For analysis, the cell is first immobilized with 10% formalin, then washed with phosphate buffered saline (PBS), subsequently stained with Sudan Red (Oil Red 0, 0.5% in 60% isopropanol) for 10 minutes, and finally washed with secondary water, and then the fat accumulation state is observed with a microscope.

Enzyme-linked immunosorbent assay (ELISA) is also used for quantitative analysis in Experimental Example 2.

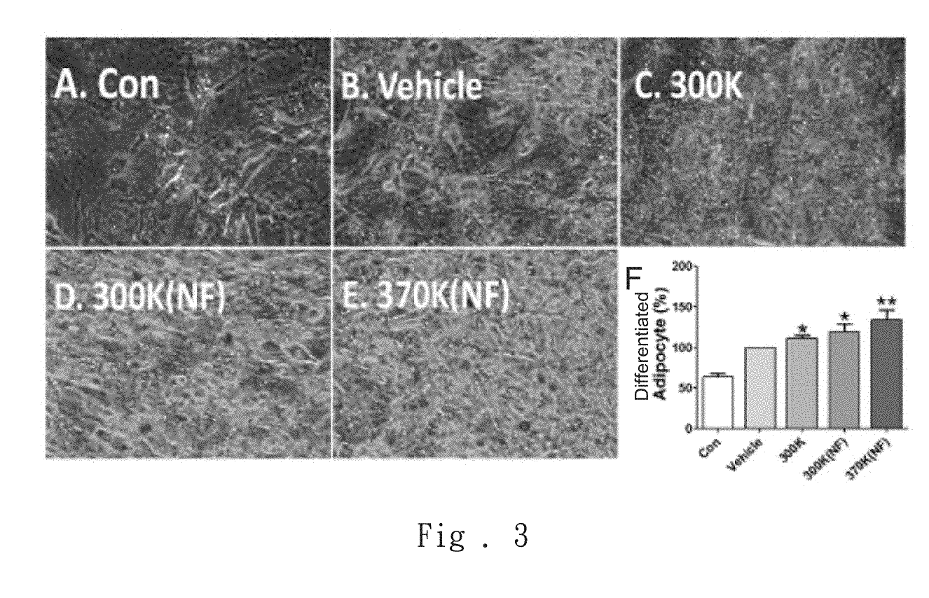

The experimental results refer to FIG. 3, which are a control group (Con) in which the 3T3-L1 pre-adipocyte is undifferentiated, a vehicle group in which only a solvent is added, 300K(NF) is treated with the drinking water composition for improving the metabolism in the first embodiment, and 370K(NF) is treated with the drinking water composition for improving the metabolism in the second embodiment.

Compared with the control group (Con) and the vehicle group, the 3T3-L1 pre-adipocyte is treated with the drinking water composition for improving the metabolism (corresponding to 300K(NF) of FIG. 3) in the first embodiment and the drinking water composition for improving the metabolism (corresponding to 370K(NF) of FIG. 3) in the second embodiment, and it may be observed under the microscope that the area which is stained red with Sudan Red 5B increases, which represents an increasing in the number of adipocytes.

As can be seen from Experimental Example 2, the drinking water composition for improving the metabolism according to the present invention effectively promotes the differentiation of the 3T3-L1 pre-adipocyte into the adipocyte.

### Experimental Example 3: The Drinking Water Composition for Improving the Metabolism According to the Present Invention Activates PPARα, PPARγ, and GLUT4 Simultaneously

The 3T3-L1 pre-adipocyte and the 3T3-L1 adipocyte are inoculated in a 6-well plate containing a culture solution respectively and grow overnight, and are collected with icy phosphate buffered saline (PBS) after 14 days of the addition of the drinking water composition for improving the metabolism.

In a reverse transcription polymerase chain reaction (RT-PCR) experiment, total RNA is extracted using a total RNA extraction kit (Amersham Pharmacia, UK), and total RNA is quantitated to 5 μg with a spectrophotometer. Total RNA is reversely transcribed into cDNA using oligo d(T), then added with forward primers and reverse primers of cDNA, and analyzed with a RT-PCR Bead Kit (Amersham Pharmacia, UK). Subsequently, the RNA is amplified into cDNA by using a gene amplification PCR system and photographed under a UV box using a 1.5% tetrabromoethane (TBE) agarose gel with ethidium bromide electrophoresis analysis.

The test genes and primers used in this experiment are shown in detail in Table 2:

TABLE 2

| | | |
|---|---|---|
| β-actin | Forward | 5′-GGCCAACCGTGAAAAGATG-3′ |
| | primer | (SEQ ID NO: 1) |
| | Reverse | 5′-GGATCTTCATGAGGTAGTCTGTC-3′ |
| | primer | (SEQ ID NO: 2) |
| PPARα | Forward | 5′-GCCATCTTCACGATGCTGTCCTCC-3′ |
| | primer | (SEQ ID NO: 3) |
| | Reverse | 5′-GTAGATCTCTTGCAACAGTGGGTGC-3′ |
| | primer | (SEQ ID NO: 4) |
| PPARγ | Forward | 5′-GGATTCATGACCAGGGAGTTCCTC-3′ |
| | primer | (SEQ ID NO: 5) |
| | Reverse | 5′-GCGGTCTCCACTGAGAATAATGAC-3′ |
| | primer | (SEQ ID NO: 6) |
| GLUT-4 | Forward | 5′-CTCATGGGCCTAGCCAATG-3′ |
| | primer | (SEQ ID NO: 7) |
| | Reverse | 5′-GGGCGATTTCTCCCACATAC-3′ |
| | primer | (SEQ ID NO: 8) |

PPAR (peroxisome proliferators activated receptor) family comprises a total of three kinds of patterns, i.e., PPARα, PPARβ, PPARγ, which present and reflect different physiological functions in different tissues. PPARα is mainly presented in the liver, skeletal muscle and heart. PPARγ is mainly presented in fat tissues. PPAR is an important mechanism for regulating the body's metabolic mechanisms. For example, PPAR will assist in the breakdown of fat to produce heat when starved, and assist in the storage of sugars in a fat form when stuffed.

GLUT4 is the most important glucose transporter in adipocytes and muscle cells. Insulin-mediated GLUT4 membrane transport is essential for the maintenance of the body's blood glucose balance. Studies have shown that GLUT4 transport impairment is often manifested in patients with insulin resistance or with type 2 diabetes.

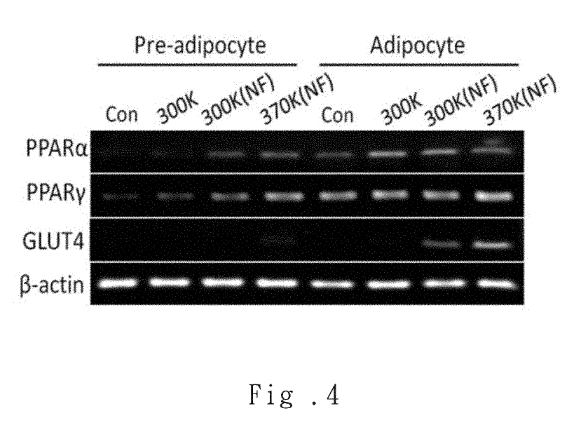

With reference to FIG. 4, the group of the 3T3-L1 pre-adipocyte which is not treated with the drinking water composition for improving the metabolism is used as the control group (Con), and it may be observed that in the group (corresponding to the 300K(NF) of FIG. 4) treated with the drinking water composition for improving the metabolism in the first embodiment and the group (corresponding to the 370K(NF) group of FIG. 4) treated with the drinking water composition for improving the metabolism in the second embodiment, the expression of PPARα and PPARγ increase. In the group treated with the drinking water composition for improving the metabolism in the second embodiment, the GLUT4 expression also slightly increases than that of the control group. However, the above trend is more obvious in the group of the 3T3-L1 adipocyte. It is represented that the drinking water composition for improving the metabolism according to the present invention activates PPARα, PPARγ, and GLUT4 simultaneously, has a positive effect on the fat storage capacity and the glucose metabolism capacity, and has the potential to be used for patients with type 2 diabetes.

From the above, it is experimentally found that the drinking water composition for improving the metabolism provided by the present invention which comprises magnesium, sodium, potassium, calcium and at least one of trace elements selected from the group consisting of chromium, boron, silicon, zinc, selenium, molybdenum, iodine and combinations thereof, in specific proportions, can not only activate the expression of blood glucose regulation genes to have a potential to lower the blood sugar level and a positive impact on the fat storage capacity of cells, but also increase the fat storage capacity of the adipocytes.

Especially for type 2 diabetics, adipocytes of these patients are unable to store fat efficiently due to insulin resistance causing from the insufficiency of adipocytes, resulting in excessive accumulation of fat in blood and peripheral tissues. Therefore, if the drinking water composition for improving the metabolism according to the present invention can promote the differentiation of adipocytes or enhancing the function of adipocytes to store fat and glucose, it will effectively improve the blood glucose balance in the body. Therefore, the drinking water composition for improving the metabolism according to the present invention also has the potential to be applied for diabetic patients, especially those with type 2 diabetes.

1. A drinking water composition for improving the metabolism, comprising:
magnesium ion in a weight percentage ranging from 75,000 ppm to 91,000 ppm;
sodium ion in a weight percentage ranging from 1,000 ppm to 8,000 ppm;
potassium ion in a weight percentage ranging from 500 ppm to 8,000 ppm;
calcium ion in a weight percentage ranging from 20 ppm to 65,000 ppm; and
at least one of trace elements selected from a group consisting of chromium ion, boron ion, silicon ion, zinc ion, selenium ion, molybdenum ion, iodine ion, and combinations thereof, wherein each of the trace elements independently has a weight percentage ranging from 100 ppb to 100 ppm.

2. The drinking water composition for improving the metabolism according to claim 1, wherein the drinking water composition for improving the metabolism has a magnesium hardness ranging from 250 mg/L to 5,000 mg/L, and the magnesium hardness is defined as magnesium ion concentration× 4.1.

3. The drinking water composition for improving the metabolism according to claim 1, wherein the weight percentage of each of the trace elements ranges from 200 ppb to 25 ppm.

4. The drinking water composition for improving the metabolism according to claim 1, wherein the trace elements are selected from a group consisting of chromium ion, zinc ion, selenium ion, and combinations thereof.

5. The drinking water composition for improving the metabolism according to claim 1, wherein the drinking water composition is prepared by concentrating deep sea water having a depth below the sea level ranging from 200 m to 1,500 m.

6. The drinking water composition for improving the metabolism according to claim 1, wherein the drinking water composition activates PPARα, PPARγ, and GLUT4 simultaneously, promotes the differentiation of an adipocyte and has an effect of increasing the fat storage capacity of the adipocyte.

7. (canceled)

8. (canceled)

9. (canceled)

10. (canceled)


SEQUENCE LISTING (8 sequences)

SEQ ID NO: 1 - DNA, 19 bp, Artificial Sequence
  Synthesised forward primer of beta-actin
ggccaaccgtgaaaagatg

SEQ ID NO: 2 - DNA, 23 bp, Artificial Sequence
  Synthesised reverse primer of  beta-actin
ggatcttcatgaggtagtctgtc

SEQ ID NO: 3 - DNA, 24 bp, Artificial Sequence
  Synthesised forward primer of PPAR alpha
gccatcttcacgatgctgtcctcc

SEQ ID NO: 4 - DNA, 25 bp, Artificial Sequence
  Synthesised reverse primer of PPAR alpha
gtagatctcttgcaacagtgggtgc

SEQ ID NO: 5 - DNA, 24 bp, Artificial Sequence
  Synthesised forward primer of PPAR gamma
ggattcatgaccagggagttcctc

SEQ ID NO: 6 - DNA, 24 bp, Artificial Sequence
  Synthesised reverse primer of PPAR gamma
gcggtctccactgagaataatgac

SEQ ID NO: 7 - DNA, 19 bp, Artificial Sequence
  Synthesised forward primer of GLUT-4
ctcatgggcctagccaatg

SEQ ID NO: 8 - DNA, 20 bp, Artificial Sequence
  Synthesised reverse primer of GLUT-4
gggcgatttctcccacatac